(19) United States
(12) Patent Application Publication
(10) Pub. No.: US 2026/0166233 A1
(43) Pub. Date: Jun. 18, 2026

(54) DEVICE FOR NASAL DELIVERY OF FLUID PRODUCT

(71) Applicant: APTAR FRANCE SAS, LE NEUBOURG (FR)

(72) Inventors: Bruno BIZET, SANCÉ (FR); Vincent CHAMARANDE, MÂCON (FR); Aurelie GOUJON BROYER, MANZIAT (FR)

(73) Assignee: APTAR FRANCE SAS, LE NEUBOURG (FR)

(21) Appl. No.: 18/714,771

(22) PCT Filed: Dec. 7, 2022
(86) PCT No.: PCT/FR2022/052269

§ 371 Date: May 30, 2024

(30) Foreign Application Priority Data
Dec. 10, 2021 (FR) FR2113279

(51) Int. Cl. A61M 5/32; B05B 1/34

(52) U.S. Cl. CPC A61M 5/3286; B05B 1/3436

(57) ABSTRACT

Device for delivery of fluid product, having a reservoir containing a dose of fluid product, a system for generating a flow of compressed gas, suitable for generating a flow of compressed gas upon actuation, and an intermediate part connecting the reservoir to the system for generating a flow of compressed gas, and having an actuation mechanism for actuating the device. The reservoir has a movable or deformable part suitable for expelling the fluid product out of the reservoir, the movable or deformable part being actuated by the flow of compressed gas generated upon the actuation, in such a way that the flow of compressed gas does not come into contact with the fluid product.

The present invention relates to a device for nasal delivery of fluid product, and more specifically to a device for dispensing a dose of pharmaceutical fluid product, in particular liquid, contained in a vessel, into a nostril with the aid of a flow of pressurized gas.

The documents WO 99/46055 and WO 02/45866 disclose nasal dispensing devices in which the fluid product contained in the reservoir is entrained by a flow of pressurized air created by an air blaster which is actuated manually by the user. The document WO 2020/154182 proposes replacing the manual air blaster with a reservoir of compressed gas which is opened during actuation in order to dispense the dose of fluid product. The device comprises a double-pointed needle, and when the user presses on the bottom of the reservoir, the needle will pierce the fluid product reservoir on the one hand and the compressed gas reservoir on the other hand, thus enabling the compressed gas to entrain and dispense the dose of fluid product.

The document GB 2 536 259 describes another prior art device, comprising a reservoir containing a compressed propellant gas.

These known devices suffer from some disadvantages. In particular, the dosing accuracy of devices that mix air or gas with the fluid product is not optimal, because the expulsion of the complete dose is not guaranteed with this type of device. In addition, using a compressed propellant gas is expensive and potentially harmful to the environment. In addition, such devices, which generally comprise metal and plastic, are more complicated to recycle.

The aim of the present invention is to provide a device for dispensing fluid product nasally which does not suffer from the aforementioned disadvantages.

In particular, the aim of the present invention is to provide a device of this type which guarantees the expulsion of all of the dose of fluid product.

The aim of the present invention is also to provide a device of this type which uses a flow of pressurized gas in order to dispense the fluid product, without the pressurized gas coming into contact with said fluid product.

The aim of the present invention is additionally to provide a device of this type which encourages the delivery of fluid product to parts which are remote from the nostril, in particular the ethmoid sinuses.

The aim of the present invention is additionally to provide a device of this type which is free from compressed propellant gas which is potentially harmful to the environment.

The aim of the present invention is also to provide a device of this type which is simple and inexpensive to manufacture, to assemble, to fill and/or to use.

Thus, the present invention concerns a device for dispensing fluid product, comprising a dispensing head the axial end of which comprises a dispensing orifice, a reservoir containing a dose of fluid product, a system for generating a flow of compressed gas adapted to generate a flow of compressed gas during actuation, and an intermediate portion connecting said reservoir to said system for generating a flow of compressed gas and comprising actuating means for actuating said device, said reservoir comprising a mobile or deformable portion adapted to expel the fluid product out of said reservoir, said mobile or deformable portion being actuated by said flow of compressed gas generated during the actuation, in a manner such that said flow of compressed gas does not come into contact with said fluid product, said system for generating a flow of compressed gas being formed by an air blaster comprising an air chamber in which an air piston slides, loading means being provided in order to displace said air piston in said air chamber in order to compress the air contained in said air chamber.

Advantageously, said air chamber comprises an opening shut off by a shutter, said shutter being adapted to be displaced and/or to be deformed during the actuation in order to open said opening, thereby generating a flow of compressed air.

Advantageously, said loading means are formed by a cover which is pivotably mounted on said device in a manner such that when said cover is displaced from a closed position towards an open position, said air piston is displaced in order to compress the air contained in said air chamber.

In accordance with a first advantageous variational embodiment, said reservoir comprises a hollow cylindrical body containing the fluid product and provided with an upper opening and a lower opening, said upper opening being connected to said dispensing orifice, and said lower opening being closed by a piston which is slidably mounted in said hollow cylindrical body, said piston forming said mobile or deformable portion of the reservoir, in a manner such that the fluid product is dispensed through said dispensing orifice when said piston slides in said hollow cylindrical body during the actuation.

In accordance with a second advantageous variational embodiment, said reservoir comprises a deformable membrane or pouch provided with an upper opening which is connected to said dispensing orifice, said deformable membrane or pouch forming said mobile or deformable portion of the reservoir, in a manner such that the fluid product is dispensed through said dispensing orifice when said deformable membrane or pouch is deformed during the actuation.

Advantageously, said actuating means comprise a lateral actuating button which is radially displaceable with respect to said dispensing head between a rest position and an actuation position.

Advantageously, said actuating means comprise an axial actuating element which is axially displaceable with respect to said dispensing head between a rest position and an actuation position.

Advantageously, said intermediate portion comprises opening means which, during the actuation, enable the compressed gas to be released.

In accordance with a first advantageous variational embodiment, said opening means comprise an apertured plate which is laterally displaceable together with a lateral actuating button, said plate shutting off an outlet of said system for generating a flow of compressed gas before actuation, and after actuation, an aperture of said apertured plate becomes positioned facing said opening, letting out the flow of compressed gas.

In accordance with a second advantageous variational embodiment, said opening means comprise a push element adapted to push a bead shutting off an outlet of said system for generating a flow of compressed gas before actuation.

In accordance with a third advantageous variational embodiment, said opening means comprise a piercing element adapted to pierce a membrane shutting off an outlet of said system for generating a flow of compressed gas before actuation.

Advantageously, said piercing element is fixed with respect to said reservoir.

In a variation, said piercing element is mobile with respect to said reservoir.

Advantageously, said fluid product is a drug in liquid form.

These features and advantages and others will become more clear in the detailed description below, made with reference to the accompanying drawings, given by way of non-limiting examples, and in which:

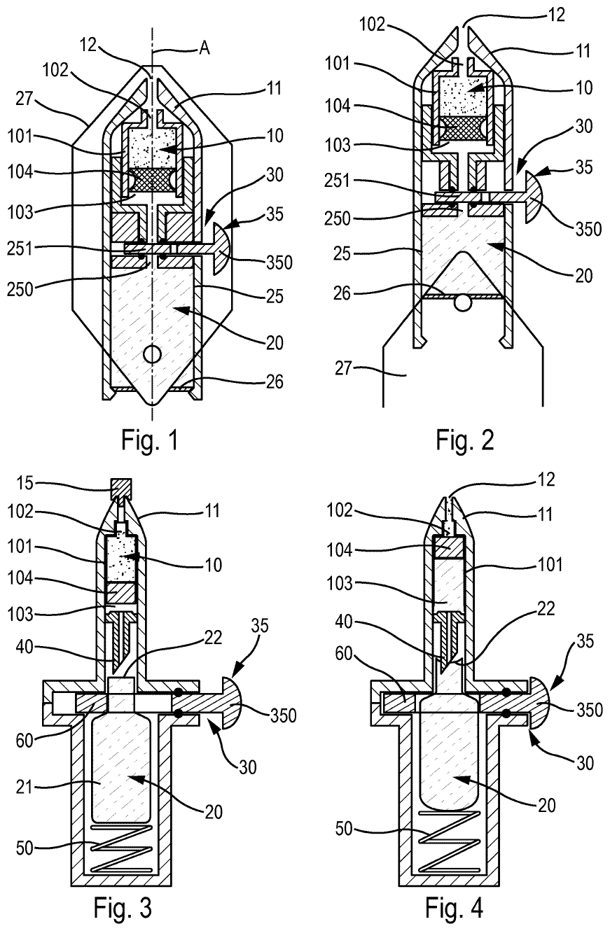

FIG. 1 is a diagrammatic sectional view of a device for dispensing fluid product in accordance with a first advantageous embodiment, before use,

FIG. 2 is a view similar to that of FIG. 1, after loading the air blaster and before actuation,

FIG. 3 is a diagrammatic sectional view of a device for dispensing fluid product in accordance with a second advantageous embodiment, before actuation,

FIG. 4 is a view similar to that of FIG. 3, after actuation,

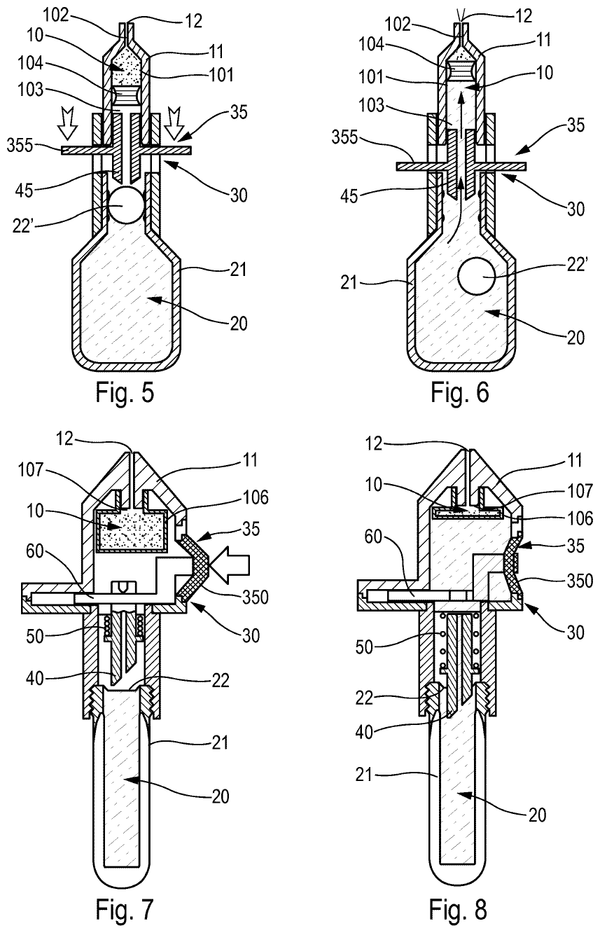

FIG. 5 is a diagrammatic sectional view of a device for dispensing fluid product in accordance with a third advantageous embodiment, before actuation,

FIG. 6 is a view similar to that of FIG. 5, after actuation,

FIG. 7 is a diagrammatic sectional view of a device for dispensing fluid product in accordance with a fourth advantageous embodiment, before actuation, and

FIG. 8 is a view similar to that of FIG. 7, after actuation,

In the description below, the terms “upper” and “lower” should be understood to be with respect to the upright position of the device as shown in the figures. The terms “axial” and “radial” refer to the longitudinal central axis A of the device, shown in FIG. 1.

The device comprises three main portions, namely a reservoir 10 containing a dose of fluid product to be dispensed, a system for generating a flow of compressed gas 20 adapted to generate a flow of compressed gas during actuation in order to expel the dose of fluid product during actuation, and an intermediate portion 30 connecting the reservoir 10 to the system for generating a flow of compressed gas 20 and enabling the actuation of the device.

The reservoir 10 is integral with a dispensing head 11 with an axially elongated shape, the axial end of which comprises a dispensing orifice 12, and which is intended to be inserted into the nostril of a user. The reservoir 10 may be formed directly in the dispensing head 11, as shown in the example of FIGS. 5 and 6. In a variation, as can be seen in FIGS. 1 to 4 and 7, 8, the reservoir 10 may be formed separately from the dispensing head 11, by being inserted into it and/or mounted in it.

Optionally, a spray profile may be provided at the dispensing orifice 12 in order to carry out dispensing of the fluid product in the form of a spray. However, in order to reach more remote parts of the nose, such as the ethmoid sinuses, it is generally desirable to limit the lateral dispersion of the fluid product, and a very wide spray would not be ideal in this case. A distribution in the form of a jet may therefore be advantageous.

The dispensing orifice 12 may be closed by a removable plug 15 which is removed just before using the device, as can be seen in FIG. 3. In the example of FIG. 1, a cover 27 closes and protects the dispensing orifice 12 before use.

The fluid product is preferably a drug in liquid form, but fluid products with greater or lesser viscosity could also be used.

In accordance with the invention, the reservoir 10 comprises a mobile or deformable portion suitable for expelling the fluid product from the reservoir, said mobile or deformable portion being actuated by said flow of compressed gas generated during the actuation, without said flow of gas coming into contact with the fluid product.

In advantageous embodiments, which can be seen in FIGS. 1 to 6, the reservoir 10 comprises a hollow cylindrical body 101 containing the fluid product and provided with an upper opening 102 and a lower opening 103. The upper opening 102 is connected to the dispensing orifice 12, and the lower opening 103 is closed by a piston 104 which is slidably mounted in the hollow cylindrical body 101.

In these embodiments, the piston 104 forms the mobile or deformable portion of the reservoir. The fluid product, disposed in the hollow cylindrical body 101 above the piston 104, is therefore dispensed through said upper opening 102 towards said dispensing orifice 12 when the piston 104 slides in the hollow cylindrical body 101 during actuation.

In another embodiment, which can be seen in FIGS. 7 and 8, the reservoir 10 comprises a deformable membrane or pouch 106. The reservoir 10 comprises an upper opening 107 connected to the dispensing orifice 12.

During actuation, the deformable membrane or pouch 106 is deformed in order to expel the fluid product through said upper opening 107 towards said dispensing orifice 12.

In this embodiment, the deformable membrane or pouch 106 forms the mobile or deformable portion of the reservoir.

In accordance with the invention, the system for generating a flow of compressed gas 20 comprises an air blaster adapted to generate a flow of compressed air.

This air blaster comprises an air chamber 25 in which an air piston 26 slides. The air chamber 25 comprises an opening 250 shut off by a shutter 251. Loading means 27 are provided in order to displace the air piston 26 in the air chamber 25 and therefore to compress the air contained in said air chamber 25.

In the example of FIGS. 1 and 2, these loading means are formed by the pivotably mounted cover in a manner such that when the cover 27 is displaced from its closed position, which can be seen in FIG. 1, towards its open position, which can be seen in FIG. 2, the air piston 26 is displaced in order to compress the air contained in the air chamber 25.

During actuation, the shutter 251 is displaced and/or deformed in order to open the opening 250, thereby generating a flow of compressed air.

In the other embodiments of FIGS. 3 to 8, the system for generating a flow of compressed gas 20 comprises a compressed gas reservoir 21 containing compressed gas. It should be noted that this solution with compressed propellant gas does not fall within the scope of the present invention, but these embodiments of FIGS. 3 to 8 are of interest in respect of other aspects of the device, and may also be applied to a device using an air blaster, i.e. free from compressed propellant gas, which is potentially harmful to the environment.

This compressed gas reservoir 21 is shut off by closing means 22, 22′, for example a membrane 22 in the examples of FIGS. 3, 4 and 7, 8 or a bead 22′ in the example of FIGS. 5, 6.

During actuation, the closing means 22, 22′ are open, for example, the membrane 22 is pierced or the bead 22′ is expelled from its shut-off position, which releases the compressed gas contained in the compressed gas reservoir 21, thereby generating a flow of compressed gas.

The intermediate portion 30 comprises actuating means 35 in order to actuate the device.

In the examples of FIGS. 1 to 4, 7 and 8, these actuating means 35 comprise a lateral actuating button 350, which can be displaced radially with respect to the dispensing head 11 between a rest position and an actuation position.

In the example of FIGS. 5 and 6, the actuating means 35 comprise an axial actuating element 355 which can be displaced axially with respect to the dispensing head 11 between a rest position and an actuation position.

The intermediate portion 30 also comprises opening means which, during actuation, can be used to displace the shutter 251 or to open the closing means 22, 22′ in order to release the flow of compressed gas.

In the example of FIGS. 1 and 2, these opening means comprise an apertured plate 251 which can be displaced laterally together with the lateral actuating button 350. Before actuation, the plate 251 shuts off the air chamber 25, and after actuation, the aperture of the apertured plate 251 comes into a position facing the opening 250 of the air chamber 25, enabling compressed air to leave.

In the examples of FIGS. 3, 4 and 7, 8, a piercing element 40 will pierce the closing membrane 22 of the compressed gas reservoir 21. Before actuation, the membrane 22 of the compressed gas reservoir 21 is kept at a distance from the piercing element 40 by a locking member 60 which is integral with the lateral actuation button 350.

In the example of FIGS. 3 and 4, the piercing element 40 is fixed and the compressed gas reservoir 21 is urged by a spring 50 against said piercing element 40. The locking member 60 cooperates with the compressed gas reservoir 21 in order to prevent it from being displaced until the moment of actuation, which releases the locking member 60.

In the example of FIGS. 7 and 8, the compressed gas reservoir 21 is fixed and the piercing element 40 is urged by a spring 50 against said compressed gas reservoir 21. The locking member 60 cooperates with the piercing element 40 in order to prevent it from being displaced until the moment of actuation, which releases the locking member 60.

In the example of FIGS. 5 and 6, a push element 45 will push the bead 22′ of the compressed gas reservoir 21. Before actuation, the push element 45 is held at a distance from the bead 22′, for example by a spring.

When the user wishes to use the device, they place the dispensing head 11 in a nostril and press on the actuating means 35 in order to trigger the opening means which will actuate the system for generating a flow of compressed gas 20, thereby generating a flow of compressed gas.

In the examples of FIGS. 1 to 4, 7 and 8, the flow Of compressed gas will displace the piston 104 in the reservoir 10 in order to expel the dose of fluid product.

In the examples of FIGS. 5 and 6, the flow of compressed gas will deform the deformable membrane or pouch of the reservoir 10 in order to expel the dose of fluid product.

Thus, the flow of compressed gas does not become mixed with the fluid product, which encourages a better dosing accuracy.

The device in accordance with the invention improves the delivery of fluid product into the nostril, by enabling the product to reach more remote parts such as the ethmoid sinuses.

The device according to the invention is in particular suitable for use in the treatment of allergic rhinitis and central pain syndrome.

In the context of allergic rhinitis, the fluid product may be selected from the following list, given by way of non-limiting example: fluticasone, mometasone, fluticasone furoate, budesonide, beclometasone, azelastine fluticasone, azelastine, tixocortol, triamcinolone acetonide, ipratropium bromide, prednisolone, ciclesonide, flunisolide, levocabastine, azelastine fluticasone furoate, olopatadine, azelastine mometasone, mometasone olopatadine, fluticasone olopatadine.

In the context of central pain syndrome, the fluid product may be selected from the following list, given by way of non-limiting example: sumatriptan, zolmitriptan, naloxone, fentanyl, esketamine, midazolam, butorphanol, dihydroergotamine, diazepam, tapentadol, alfentanil, diamorphine, ketorolac.

The present invention has been described with reference to various embodiments and variations, but it should be understood that a person skilled in the art could provide any modifications to it without departing from the scope of the present invention as defined in the accompanying claims.

In particular, features described with reference to one embodiment could also be applied to other embodiments. Thus, for example, and in a non-limiting manner, the embodiment of FIGS. 5 and 6 could include a membrane 22 instead of a bead 22′. Similarly, the deformable pouch of the embodiment of FIGS. 7 and 8 could be replaced by a reservoir with a piston, as described in the embodiments of FIGS. 1 to 6.

Clearly, other variational embodiments may also be envisaged, without departing from the scope of the present invention as defined in the accompanying claims.

1-14. (canceled)

15. A device for dispensing fluid product, comprising a dispensing head the axial end of which comprises a dispensing orifice, a reservoir containing a dose of fluid product, a system for generating a flow of compressed gas adapted to generate a flow of compressed gas during actuation, and an intermediate portion connecting said reservoir to said system for generating a flow of compressed gas and comprising actuating means for actuating said device, said reservoir comprising a mobile or deformable portion adapted to expel the fluid product out of said reservoir, said mobile or deformable portion being actuated by said flow of compressed gas generated during the actuation, in a manner such that said flow of compressed gas does not come into contact with said fluid product, said system for generating a flow of compressed gas being formed by an air blaster comprising an air chamber in which an air piston slides, loading means being provided in order to displace said air piston in said air chamber in order to compress the air contained in said air chamber, characterized in that said actuating means comprise a lateral actuating button which is radially displaceable with respect to said dispensing head between a rest position and an actuation position.

16. The device as claimed in claim 15, in which said air chamber comprises an opening shut off by a shutter, said shutter being adapted to be displaced and/or to be deformed during the actuation in order to open said opening, thereby generating a flow of compressed air.

17. The device as claimed in claim 15, in which said loading means are formed by a cover which is pivotably mounted on said device in a manner such that when said cover is displaced from a closed position towards an open position, said air piston is displaced in order to compress the air contained in said air chamber.

18. The device as claimed in claim 15, in which said reservoir comprises a hollow cylindrical body containing the fluid product and provided with an upper opening and a lower opening, said upper opening being connected to said dispensing orifice, and said lower opening being closed by a piston which is slidably mounted in said hollow cylindrical body, said piston forming said mobile or deformable portion of the reservoir, in a manner such that the fluid product is dispensed through said dispensing orifice when said piston slides in said hollow cylindrical body during the actuation.

19. The device as claimed in claim 15, in which said reservoir comprises a deformable membrane or pouch provided with an upper opening which is connected to said dispensing orifice, said deformable membrane or pouch forming said mobile or deformable portion of the reservoir, in a manner such that the fluid product is dispensed through said dispensing orifice when said deformable membrane or pouch is deformed during the actuation.

20. The device as claimed in claim 15, in which said intermediate portion comprises opening means which, during the actuation, enable the compressed gas to be released.

21. The device as claimed in claim 20, in which said opening means comprise an apertured plate which is laterally displaceable together with a lateral actuating button, said plate shutting off an outlet of said system for generating a flow of compressed gas before actuation, and after actuation, an aperture of said apertured plate becomes positioned facing said opening, letting out the flow of compressed gas.

22. The device as claimed in claim 20, in which said opening means comprise a push element adapted to push a bead shutting off an outlet of said system for generating a flow of compressed gas before actuation.

23. The device as claimed in claim 20, in which said opening means comprise a piercing element (40) adapted to pierce a membrane shutting off an outlet of said system for generating a flow of compressed gas before actuation.

24. The device as claimed in claim 23, in which said piercing element is fixed with respect to said reservoir.

25. The device as claimed in claim 23, in which said piercing element is mobile with respect to said reservoir.

26. The device as claimed in claim 15, in which said fluid product is a drug in liquid form.
